(19) United States
(12) Patent Application Publication
(10) Pub. No.: US 2006/0047007 A1
(43) Pub. Date: Mar. 2, 2006

(54) Method for treating urinary disorders

(76) Inventors: Susan M. Danehower, New York, NY (US); Barbara Helene Korberly, Flemington, NJ (US)

Correspondence Address: PFIZER INC., PATENT DEPARTMENT, MS8260-1611, EASTERN POINT ROAD, GROTON, CT, 06340, US

(21) Appl. No.: 10/762,726

(22) Filed: Jan. 22, 2004

Related U.S. Application Data
(60) Provisional application No. 60/441,690, filed on Jan. 22, 2003.

(51) Int. Cl. A61K 31/137

(52) U.S. Cl. 514/649

(57) ABSTRACT

The present invention relates to a method, preferable an oral method, for treating urinary disorders, such as unstable or overactive bladder, while minimizing the occurrences of dry mouth, dyspepsia and reduced stream of tears. The methods of the present invention comprise orally administering to a mammal, preferably a human, a pharmaceutically effective dose of an antimuscarinic agent, such as tolterodine, when needed, whereby a symptomatic relief of urgency and/or frequency is achieved.

### FIELD OF INVENTION

The present invention relates to oral methods for treating urinary disorders such as unstable or overactive urinary bladder in a mammal while minimizing adverse events and side-effects such as the occurrence of dry mouth, dyspepsia and reduced stream of tears. These methods comprise orally administering to a mammal a pharmaceutically effective dose of tolterodine or related compounds when needed, whereby a symptomatic relief of urgency and/or frequency is achieved.

### BACKGROUND OF THE INVENTION

A substantial part (5-10%) of the adult population suffers from urinary incontinence, and the prevalence, particularly of so-called urge incontinence, increases with age. The symptoms of an unstable or overactive bladder comprise urge incontinence, urgency and urinary frequency. It is assumed that unstable or overactive bladder is caused by uncontrolled contractions of the bundles of smooth muscle fibers forming the muscular coat of the urinary bladder (the detrusor muscle) during the filling phase of the bladder. These contractions are mainly controlled by cholinergic muscarinic receptors, and the pharmacological treatment of unstable or overactive bladder has been based on muscarinic receptor antagonists.

The reason why the bladder muscle contracts inappropriately is unclear in many cases. For some people it may be due to a problem with nerve signals that run from the brain to the bladder. Surgery or childbearing sometimes causes minor nerve damage. This muscle squeezes or contracts more often than normal and at inappropriate times. Instead of staying at rest as urine fills the bladder, the detrusor contracts while the bladder is filling with urine. This causes a person to feel a sudden and sometimes overwhelming urge to urinate even when the bladder is not filled.

Overactive urinary bladder encompasses a variety of urinary disorders including overactive detrusor (detrusor instability, detrusor hyperreflexia) and sensory urgency and the symptoms of detrusor overactivity, e.g. urge incontinence, urgency and urinary frequency and LUTS (Lower Urinary Tract Symptoms including obstructive urinary symptoms such as slow urination, dribbling at the end of urination, inability to urinate and/or the need to strain to urinate at an acceptable rate or irritate symptoms such as frequency and/or urgency). Also other conditions are included, which give rise to urinary frequency, urgency and/or urge incontinence. Overactive bladder disorders also include nocturia and mixed incontinence. While overactive bladder is often associated with detrusor muscle instability, disorders of bladder function may also be due to neuropathy of the central nervous system (detrusor hyperreflexia) including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis.

A specific urinary disorder, which can be treated by the claimed method, is a dry overactive bladder, which includes frequency, urgency and nocturia.

Antimuscarinic compounds have been developed for the treatment of urinary disorders such as unstable or overactive bladder. The drug of choice has earlier been oxybutynin (marketed as, for example, Ditropan®). Typically, patients are given 5-15 mg per day for a sustained release formulation, or 5-30 mg per day of an immediate release formulation. Recently, however, an improved muscarinic receptor antagonist, tolterodine, (R)-N,N-diisopropyl-3-(2-hydroxy-5-methylphenyl)-3-phenylpropanamine, has been marketed for the treatment of unstable or overactive bladder with symptoms including urge incontinence, urinary urgency and urinary frequency. Both tolterodine and its major active metabolite, the 5-hydroxymethyl derivative of tolterodine, which significantly contributes to the therapeutic effect, have considerably less side effects than oxybutynin, especially regarding the propensity to cause dry mouth. While tolterodine is equipotent with oxybutynin in the bladder, its affinity for muscarinic receptors of the salivary gland is eight times lower than that of oxybutynin; see, for example, Nilvebrant L., et al.; European Journal of Pharmacology 327 (1997) 195-207. The selective effect of tolterodine in humans is described in Stahl, M. M. S., et al., Neurourology and Urodynamies 14 (1995) 647-65, and Bryne, N., International Journal of Clinical Pharmacology and Therapeutics, Vol. 35, No. 7 (1995) 287-295. Tolterodine is presently being sold in a number of different countries for treatment of urinary incontinence under the name Detrol®, marketed by Pharmacia (now part of Pfizer).

As mentioned above, the chemical name of tolterodine is (R)-N,N-diisopropyl-3-(2-hydroxy-5-methylphenyl)-3-phenylpropanamine. The term “related compound(s)” is meant to encompass the major, active metabolite of tolterodine, i.e. (R)-N,N-diisopropyl-3-(2-hydroxy-5-hydroxymethylphenyl)-3-phenylpropanamine; the corresponding (S)-enantiomer to tolterodine, i.e. (S)-N,N-diisopropyl-3-(2-hydroxy-5-methylphenyl)-3-phenylpropanamine; the 5-hydroxymethyl metabolite of the (S)-enantiomer, i.e. (S)-N,N-diisopropyl-3-(2-hydroxy-5-hydroxymethylphenyl)-3-phenylpropanamine; as well as the corresponding racemate to tolterodine, i.e. (R,S)-N,N-diisopropyl-3-(2-hydroxy-5-methylphenyl)-3-phenylpropanamine; and pharmaceutically acceptable salts of these compounds, as well as prodrug forms thereof (see e.g. WO99/58478). Specifically included is tolterodine L-tartrate.

Another tolterodine-related compound is fesoterodine (2-[(1R)-3-[bis(1-methylethyl)amino]-1-phenylpropyl]-4-hydroxymethylphenyl isobutyrate or alternatively R-(+)-isobutyric acid 2-(3-diisopropylamino-1-phenylpropyl)-4-hydroxymethylphenyl ester), disclosed in European patent application EP 1 077 912.

Tolterodine, its corresponding (S)-enantiomer and racemate and the preparation thereof are described in e.g. U.S. Pat. No. 5,382,600 (WO89/06644). For a description of the active (R)-5-hydroxymethyl metabolite of tolterodine (as well as the (S)-5-hydroxymethyl metabolite), it may be referred to U.S. Pat. No. 5,559,269 (WO94/11337), which also discloses that this compound is useful when urinary incontinence is treated. The (S)-enantiomer, its non-cholinergic spasmolytic activity and use in the treatment of urinary and gastrointestinal disorders are described in WO 98/03067.

The term “pharmaceutically effective amount” or “pharmaceutically effective dose”, as used herein, means the amount of antimuscarinic compound, such as tolterodine or related compounds, that will elicit the desired therapeutic effect or response, in accordance with the desired treatment regime. A preferred pharmaceutically effective amount or dose of tolterodine or related compounds is the amount that achieves symptomatic relief of urinary urgency and/or urinary frequency.

The currently marketed administration forms of tolterodine are film-coated tablets for immediate release and capsules or film coated tablets for controlled release. The immediate release tablets contain 1 mg, or 2 mg of tolterodine L-tartrate for immediate release in the gastrointestinal tract. The capsules or film-coated tablets for oral controlled release formulation for once-daily administration have a dosage of tolterodine or related compound of 2 mg or 4 mg or 6 mg. The recommended dosage is usually 2 mg twice a day for chronic use. The side effects, as earlier mentioned, such as dry mouth, are much lower than for oxybutynin, however they still exist, especially at higher dosages.

Other antimuscarinic agents include, for example, oxybutynin (J&J), darifenacin (EP 388054; Novartis), solifenacin (Y-905; Yamanouchi; Fujii, T. et al. (2000) Gen. Pharmacol. 35(2), 71-75; Ikeda, K. et al. (2002) Naunyn-Schmiedeberg's Arch Pharmacol 366, 97-103), and pharmaceutically acceptable salts and derivatives thereof.

Still further antimuscarinic agents can be found, for example, in WO 03/087096, JP2003267977, WO 03/087094, WO 03/064417, WO 03/064418, WO 03/064419, and EP733621.

### DESCRIPTION OF THE INVENTION

In the present invention it is unexpectedly found that the occurrence of dry mouth, dyspepsia and reduced stream of tears that can be associated with the high dosage of tolterodine can be minimized by administering the tolterodine or related compounds, at lower dosage to a mammal when needed; preferably, two pharmaceutically effective doses are administered daily within a dose interval of within 8-12 hours. In other words, it is found that the administration of tolterodine or related compounds, in a low dosage when needed causes less side-effects but achieves a symptomatic relief of urgency and/or frequency.

Consumers constantly require alternative methods of administration, especially when the need for medicament treatment is urgent and/or when the patient has an active life-style. Thus, the administration method of this invention will be especially beneficial in treating these above-mentioned consumers. Furthermore, from a patient lifestyle standpoint the methods of the present invention would also be more convenient than the usual earlier recommended methods of administration, requiring chronic dosing of, for example, 2×2 mg tolterodine daily permanently.

For these and other purposes, it is an object of the present invention to provide a method of administration, which method brings symptomatic relief from symptoms arising from said urinary disorder such as e.g. urinary urgency and/or frequency.

It is also an object of the present invention to provide methods of treating urinary disorder in a mammal, which methods involve alternative methods of administration and which methods are compatible with an active life-style.

One embodiment of the invention is therefore the use of an antimuscarinic agent for the manufacture of a medicament for oral administration of a pharmaceutically effective dose of the antimuscarinic agent when needed to a mammal with a urinary disorder such as unstable or overactive urinary bladder, whereby a symptomatic relief of urgency and frequency is achieved. Preferably the antimuscarinic agent is tolterodine or related compounds, or a pharmaceutically acceptable salt thereof, even more preferably, it is tolterodine L-tartrate. In other preferred embodiments of the invention, the antimuscarinic agent is selected from oxybutynin, darifenacin, solifenacin, or pharmaceutically acceptable salts or derivatives of any of these compounds.

Preferably, the mammal is a human.

Another embodiment of the invention is the use according to the invention wherein the pharmaceutically effective dose of the antimuscarinic agent, preferably tolterodine or related compounds or pharmaceutically acceptable salts thereof, most preferably tolterodine L-tartrate, is administered twice daily at an interval of 8-12 hours. Preferably, the pharmaceutically effective dose of the antimuscarinic agent is 1 mg administered as an immediate release tablet or capsule, or the pharmaceutically effective dose of the antimuscarinic agent is 1 mg or 2 mg administered as a controlled release tablet or capsule. Preferably, the pharmaceutically effective dose of the antimuscarinic agent is administered twice daily within an interval of 8 hours, 9 hours, 10 hours, 11 hours, or 12 hours. In another embodiment of the invention, the pharmaceutically effective dose of the antimuscarinic agent is 2 mg or 4 mg administered as a controlled release capsule or tablet when needed or once a day.

Another embodiment of the invention is therefore a method for treating urinary disorders such as unstable or overactive urinary bladder in a mammal, said method comprising orally administering to a mammal a pharmaceutically effective dose of an antimuscarinic agent when needed, whereby a symptomatic relief of urgency and/or frequency is achieved. Preferably the antimuscarinic agent is tolterodine or related compounds, or a pharmaceutically acceptable salt thereof; even more preferably, it is tolterodine L-tartrate. In other preferred embodiments the antimuscarinic agent is oxybutynin, darifenacin, solifenacin, or pharmaceutically acceptable salts or derivatives of any of these compounds.

In the most preferred embodiment of the method the mammal is a human.

In one preferred embodiment of the method according to the invention two pharmaceutically effective doses of the antimuscarinic agent, preferably of tolterodine or related compounds, are administered in a dose interval of within 8-12 hours.

In one preferred embodiment of the method the pharmaceutically effective dose of the antimuscarinic agent, preferably of tolterodine or related compounds, is 1 mg administered as an immediate release tablet or capsule.

In another preferred embodiment of the method the pharmaceutically effective dose of the antimuscarinic agent, preferably of tolterodine or related compounds, is 1 mg or 2 mg administered as a controlled (sustained) release tablet or capsule.

In another preferred embodiment of the method the pharmaceutically effective dose of the antimuscarinic agent, preferably of tolterodine or related compounds, is 2 mg or 4 mg administered as a controlled (sustained) release tablet or capsule.

In the most preferred embodiments of the method-said dose interval is within 8 hours, 9 hours, 10 hours, 11 hours or 12 hours.

A further aspect of the invention is a method for treating unstable or overactive urinary bladder in a mammal, said method comprising orally administering to a mammal a pharmaceutically effective dose of tolterodine or related compounds and when needed, whereby a symptomatic relief of urgency and frequency is achieved. In a preferred aspect, the pharmaceutically effective dose of tolterodine or related compounds is selected from tolterodine L-tartrate.

The invention will now be described by way of example only, not intended to be limiting the invention in any way. Methods routinely applied by the skilled person are used, in addition to those described in detail in the Examples below.

### EXAMPLE 1

The objective of a study is to evaluate the efficacy and safety of tolterodine tartrate (Detrol®) tablets 2 mg daily versus placebo, in the treatment of urinary urgency and frequency in women, in a randomized, double blind, placebo-controlled study. The patients received 1 mg tolterodine b.i.d. or placebo, and the dosage regime was one tablet orally, every 8-12 hours, not to exceed 2 tablets daily. The treatment duration was 10 days, after a 5 day pretreatment baseline phase. 1315 hundred women were included in the study, and were randomized to either the tolterodine or placebo group. All of the subjects were eligible for intent-to-treat (ITT) and safety analysis. 1077 of the randomized subjects were included in the per-protocol analysis.

Females with symptoms of urinary urgency, defined as having to stop the current activity and go immediately to the bathroom at least once a day (severe) or able to finish a task but go right to the bathroom at least twice a day (moderate), were included. Frequency was defined as >7 micturitions per 24 hours. The mean age was 48 years. About 83% of the subjects were Caucasian. The two treatment groups were comparable with respect to the demographic data.

Women were asked to complete a diary recording the time of awakening; number and severity of urgency of each micturition, and the number of episodes of incontinence throughout the day as well as, time when tablets taken. The women were directed to begin using the study drug after 5 days (baseline, pretreatment period): 1 tablet every 8-12 hours not to exceed 2 tablets per day.

Thus, this study was designed to evaluate the efficacy and safety of tolterodine 1 mg twice daily, every 8-12 hours, compared to placebo, in women with urinary urgency and frequency. The primary efficacy endpoints were:
1. Subject perception of improvement in symptoms of urgency after five days of treatment, vs. baseline period, using a three-point categorical scale (Chi Square analysis):
2. Mean of daily average severity of urgency for urgent episodes (urgency rating score of at least 1) for the 5-day baseline period vs. first five days of treatment.

A five-point categorical scale was used to assess the severity of urgency experienced at each micturition, ranging from 0 (no discomfort) to 4 (very severe).

The secondary efficacy endpoints were:
1. Average number of urgency episodes per day per subject for the 5-day baseline period vs. first five days of treatment.
2. Average urgency score (severity of urgency) per subject for micturition episodes accompanied by urgency (a rating score of at least 1) for the 5-day baseline period vs. first day of treatment.
3. Average number of micturitions per day per subject for the 5-day baseline period vs. first five days of treatment.

Subject perception of improvement in symptoms or urgency after ten days of treatment, vs. baseline period, using a three-point categorical scale (Chi Square analysis).

The sudden urge to urinate accompanied by frequency of urination is a problem for many adults. The symptoms of urgency are most problematic. Therefore, the primary variables examined the subjective perception of improvement in urgency as well as the severity of urgency in subjects treated with tolterodine 1 mg b.i.d. In addition, the frequency of urination was examined as this is a standard assessment tool commonly used to assess the efficacy of drugs for the treatment of overactive bladder syndrome.

Two populations were analyzed for efficacy: the intent-to-treat (ITT) and the per-protocol populations. The former comprised all individuals randomized to treatment. The latter consisted of those individuals who complied with the protocol with regard to eligibility, visit schedule, diary completion and treatment schedule. All randomized subjects who took study medication were included in the safety analyses. Incidence of adverse events was tabulated for all randomized subjects.

The data obtained from this study were assessed by descriptive statistics, as well as by appropriate non-parametric and parametric (ANOVA) comparisons of the two treatment groups for efficacy and safety. More specifically, subject perception of improvement in symptoms of urgency was analyzed using Cochran-Mantel-Haenszel (CMH) test, controlling for center effect. Unless otherwise stated, change from baseline variables was analyzed via an ANOVA model including treatment and center effects. Treatment-by-center effects were assessed by adding treatment-by-center term to the original model. If treatment-by-center interaction was detected at 0.05 level, the interaction would be assessed. If the source of the interaction could be traced to one or two aberrant centers, then an additional analysis would be performed excluding those centers.

Results:

The tolterodine group had significantly higher baseline severity compared to the placebo group for three efficacy variables: mean of daily average severity of urgency at micturition score (1.99 vs. 1.94, p=0.041), mean of daily average severity for urgent episodes score (2.10 vs. 2.06, p=0.046), average daily number of pads used (0.62 vs. 0.49, p=0.05). To account for these imbalances, it was decided post hoc to include the baseline effect in the analysis model.

The Efficacy Results:

Subject perception of improvement in symptoms of urgency on Day 5 was one of the two primary efficacy variables. The tolterodine group had a statistically significantly higher percentage of subjects reporting symptom improvement on Day 5 compared to placebo. Similar results were observed on Day 10.

Severity of urgency for all micturitions: The tolterodine group had a statistically significantly greater decrease in severity of urgency, compared to placebo, for all analyzed time points, including day 1.

Severity of urgency for urgent episodes: The tolterodine group had a statistically significantly greater decrease in severity of urgency for urgent episodes, compared to placebo, for all analyzed time points, including day 1. For the primary time point of interest (days 1-5), the improvement from baseline was 0.27 for the tolterodine group and 0.19 for the placebo group.

Number of micturitions/day: The tolterodine group had a statistically significantly greater decrease in the number of micturitions, compared to placebo, for all analyzed time points. For example for days 1-5, a reduction from baseline of 1.59 was achieved in the tolterodine-treated group, whereas only a reduction from baseline of 1.26 was achieved in the placebo group (N=660 for tolterodine, N=655 for placebo).

Number of urgent episodes/day: The tolterodine group had a statistically significantly greater reduction in the number of urgent episodes, compared to placebo, for all analyzed time points.

To summarize, the tolterodine group had, compared to placebo, statistically significant:
Greater percentage of subjects experiencing improvement;
Lower urgency severity scores;
Fewer number of urgent episodes per day;
Fewer number of micturitions per day.

Overall, these results demonstrate that tolterodine 1 mg b.i.d daily is effective in treating symptoms of urinary urgency and frequency with acute treatment. Importantly, the perceived sense of urgency decreased significantly after the first day of treatment.

The Safety Results:

Seventy-one (10.8%) subjects in the tolterodine group and 70 (10.7%) subjects in the placebo group reported adverse events. Of these, thirty-two (4.8%) subjects in the tolterodine group and 31 (4.7%) subjects in the placebo group had adverse events that were considered related to study medication by the investigators.

Three subjects in the tolterodine group and one subject in the placebo group reported serious AEs. Three subjects in the tolterodine group and four subjects in the placebo group discontinued from the study due to AEs. AEs related to gastrointestinal disorders were the most commonly reported: 37 (5.6%) tolterodine subjects and 32 (4.9%) placebo subjects. AEs related to nervous system disorders were the next most commonly reported: 20 (3.0%) subjects in the tolterodine group and 13 (2.0%) subjects in the placebo group.

Conclusion

This study demonstrates that tolterodine 1 mg b.i.d. is effective in decreasing the severity of the sensation of urgency and the frequency of micturitions in the population studied. Additionally, significantly more subjects in the tolterodine group, compared to the placebo group, reported improvement in their symptoms. Importantly, improvements were seen by the first day of treatment and continued throughout the treatment period.

This suggests that a woman experiencing urinary urgency and/or frequency can expect to see a beneficial impact of tolterodine treatment with acute use. This is significant as it allows those who experience these symptoms to treat only as needed, when symptoms are or are anticipated to be bothersome (e.g. a restroom is not readily assessable or frequent trips to the restroom are not feasible). The rapid onset of therapeutic benefit allows for treatment to be instituted on an “as needed” (prn) basis, based on a person's desire to control symptoms.

### EXAMPLE 2

### Evaluation of Other Antimuscarinic Agents

Similar studies as described in Example 1 can be performed with other antimuscarinic agents, such as darifenacin, solifenacin, fesoterodine, or pharmaceutically acceptable salts or derivatives of any of these compounds. The skilled person will be able to select suitable pharmaceutically effective doses, for example from results obtained in standard long-term clinical trials.

1. Use of an antimuscarinic agent for the manufacture of a medicament for oral administration of a pharmaceutically effective dose of the antimuscarinic agent when needed to a mammal with unstable or overactive urinary bladder, whereby a symptomatic relief of urgency and/or frequency is achieved.

2. The use as claimed in claim 1, wherein the antimuscarinic agent is one or more compounds selected from tolterodine and related compounds.

3. The use according to claim 2, wherein the compound is tolterodine or a pharmaceutically acceptable salt thereof.

4. The use according to claim 1, wherein the antimuscarinic agent is selected from oxybutynin, darifenacin, solifenacin, and pharmaceutically acceptable salts and derivatives thereof.

5. The use according to any of claims 1 to 4, wherein the mammal is a human.

6. The use according to claim 5, wherein the pharmaceutically effective dose is 2 mg or 4 mg of the antimuscarinic agent, administered as a controlled release tablet or capsule.

7. The use according to claim 5 wherein two pharmaceutically effective doses of the antimuscarinic agent are administered daily at an interval of 8-12 hours.

8. The use according to claim 7, wherein the pharmaceutically effective dose is 1 mg of the antimuscarinic agent, administered as an immediate release tablet or capsule.

9. The use according to claim 7, wherein the pharmaceutically effective dose is 1 mg or 2 mg of the antimuscarinic agent, administered as a controlled release tablet or capsule.

10. The use according to any of claims 1-5 or claims 7-9, wherein the pharmaceutically effective doses of the antimuscarinic agent are taken within the interval of 8 hours.

11. The use according to any of claims 1-5 or claims 7-9, wherein the pharmaceutically effective doses of the antimuscarinic agent are taken within the interval of 9 hours.

12. The use according to any of claims 1-5 or claims 7-9, wherein the pharmaceutically effective doses for the antimuscarinic agent are taken within the interval of 10 hours.

13. The use according to any of claims 1-5 or claims 7-9, wherein the pharmaceutically effective doses of the antimuscarinic agent are taken within the interval of 11 hours.

14. The use according to any of claims 1-5 or claims 7-9, wherein the pharmaceutically effective doses of the antimuscarinic agent are taken within the interval of 12 hours.

15. A method for treating unstable or overactive urinary bladder in a mammal, said method comprising orally administering to a mammal a pharmaceutically effective dose of an antimuscarinic agent and when needed, whereby a symptomatic relief of urgency and/or frequency is achieved.
